(19) United States
(12) Patent Application Publication
(10) Pub. No.: US 2013/0225845 A1
(43) Pub. Date: Aug. 29, 2013

(54) PROCESS FOR PREPARATION OF ESTRADIOL VALERATE AND A NOVEL CRYSTALLINE FORM A OF ESTRADIOL DIVALERATE

(71) Applicants: Sachin Arun Sasane, Pune (IN); Vijay Ashok Ahire, Pune (IN); Rajesh Vyas, Pune (IN); Nandu Baban Bhise, Pune (IN); Girij Pal Singh, Pune (IN)

(72) Inventors: Sachin Arun Sasane, Pune (IN); Vijay Ashok Ahire, Pune (IN); Rajesh Vyas, Pune (IN); Nandu Baban Bhise, Pune (IN); Girij Pal Singh, Pune (IN)

(73) Assignee: LUPIN LIMITED, Mumbai, Maharashtra (IN)

(21) Appl. No.: 13/883,593

(22) PCT Filed: Oct. 28, 2011
(86) PCT No.: PCT/IB2011/002558

§ 371 Date: May 6, 2013

(30) Foreign Application Priority Data
Nov. 4, 2010 (IN) 1233/KOL/2010

(51) Int. Cl. C07J 1/00

(52) U.S. Cl. CPC C07J 1/0074
USPC 552/625

(57) ABSTRACT

The present invention relates to the process for the preparation of estradiol valerate (I) which involves isolation of crystalline estradiol divalerate (III) by crystallization from an alcoholic solvent.

### FIELD OF THE INVENTION

The present invention relates to an improved process for the preparation of estradiol valerate (I) with high purity. The invention also relates to the process for the preparation of novel crystalline Form A of estradiol divalerate (III) and process for its preparation.

### BACKGROUND OF THE INVENTION:

Estradiol valerate (I) is a female estrogen hormone. It works by replacing natural estrogens in a woman who can no longer produce enough estrogen. It works for advanced prostate cancer by antagonizing male hormones. It is also used to prevent bone loss (osteoporosis) in those who cannot take non-estrogen drugs.

Estradiol valerate (I) is designated chemically as estra-1,3,5 (10)-triene-3,17-diol(17β)-, 17-pentanoate.

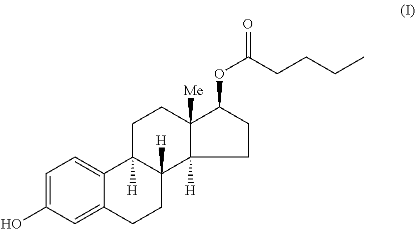

Estradiol valerate was first disclosed in product patent U.S. Pat. No. 2,160,555. The product patent also describes preparation of estradiol valerate as depicted in scheme-I, by reaction of n-valeric anhydride on estradiol (II) in the presence of pyridine to give estradiol divalerate (III) as oil.

The oily estradiol divalerate (III) is treated with potassium carbonate in methanol to give estradiol monovalerate (I) which is crystallized from methanol-water mixture.

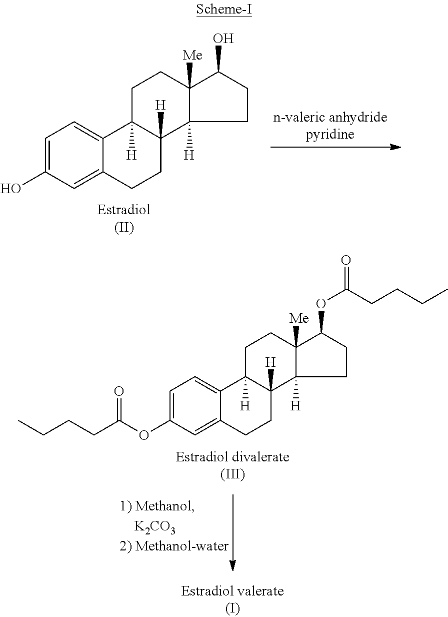

The patent U.S. Pat. No. 2,205,627 describes the preparation of estradiol valerate by reaction of estradiol (II) with valeric anhydride in the presence of pyridine to give estradiol divalerate (III) as oil which is further purified by distillation under high vacuum and then converted to estradiol valerate (I) by using potassium carbonate.

The prior art methods discussed above suffer from the following disadvantages:
a) the intermediate estradiol divalerate (III) is obtained as an oil, which makes it difficult to handle.
b) prior art method uses distillation under very high vacuum such as 0.01 mm of Hg for the purification of estradiol divalerate which makes it not only laborious but unfavorable on plant scale as well, since it is difficult to maintain high vacuum and is time consuming.
c) Poor yields of estradiol valerate (I) since oily compound is difficult to distil out completely which results in loss of yield of pure estradiol divalerate (III).
d) Lower purity of estradiol divalerate (III).

It is quite likely that estradiol valerate when prepared from impure estradiol divalerate would not meet with the pharmaceutically acceptable quality. Therefore, there is, an unfulfilled need to provide industrially feasible process for the preparation of estradiol valerate with higher purity. The present invention is directed for the same and provides estradiol valerate in purity≧99.4%.

It has been discovered that a substance can exist in different polymorphic crystalline forms which differ from each other by their stability, physical properties, spectral characteristics and the process for their preparation. One of the polymorph might have more stability than the other and hence suitable for storage and handling. The present invention describes a new crystalline form of estradiol divalerate named Form A.

### SUMMARY OF THE INVENTION:

The present invention provides an improved process for the preparation of estradiol valerate (I) that comprises the following steps:
reaction of estradiol (II) with n-valeric anhydride or n-valeryl chloride in the presence of base to obtain estradiol divalerate (III),
(ii) crystallization of estradiol divalerate (III) from alcoholic solvent to obtain crystalline estradiol divalerate (III), and
(iii) conversion of crystalline estradiol divalerate (III) to estradiol valerate (I),

The present invention also provide a novel crystalline form (A) of estradiol divalerate.

### DESCRIPTION OF THE DRAWINGS

FIG. 1: X-ray powder diffractogram (XRPD) for crystalline form A of estradiol divalerate.

FIG. 2: IR spectrum for crystalline form A of estradiol divalerate.

### DETAILED DESCRIPTION OF THE INVENTION:

The present invention provides an improved process for the preparation of estradiol valerate (I), that comprises of:
(i) reaction of estradiol (II) with n-valeric anhydride or n-valeryl chloride in the presence of base to obtain estradiol divalerate (III),
(ii) crystallization of estradiol divalerate (III) from alcoholic solvent to obtain crystalline estradiol divalerate (III), and
(iii) conversion of crystalline estradiol divalerate (III) to estradiol valerate (I),

The synthetic scheme of the process of present invention is shown in scheme II

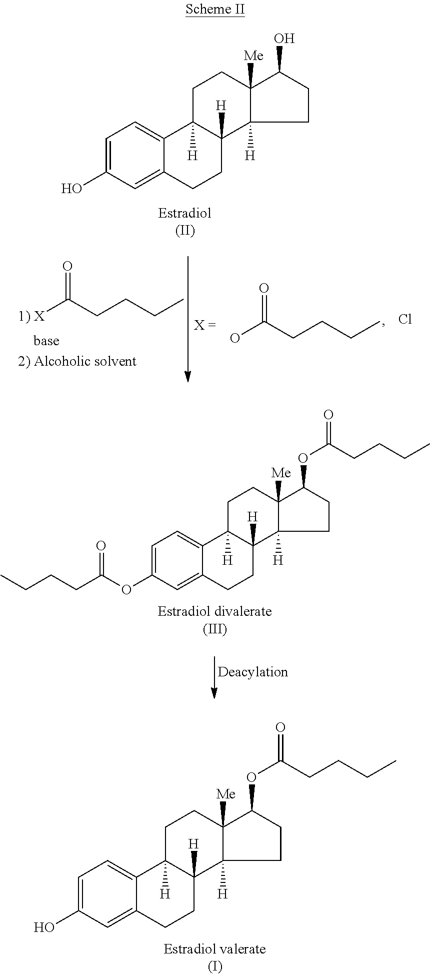

The process of step (i) involves reaction of estradiol (II) with n-valeric anhydride or n-valeryl chloride in the presence of base to give estradiol divalerate (II).

The base used in step (i) is selected from a group of organic or inorganic bases. The inorganic bases are selected from group of carbonates or hydroxides of alkali earth metals like sodium hydroxide, potassium hydroxide, sodium carbonate, potassium carbonate, sodium bicarbonate and potassium bicarbonate. The organic base is selected from a group of pyridine, N-methyl morpholine, N-methyl pyrrolidine, tertiary alkyl amine such as triethyl amine, tertiary butyl amine etc. The most preferred base is pyridine.

The reaction can be carried out in organic solvent or mixture of organic solvent and water. The organic solvents can be selected from aromatic hydrocarbons like benzene, toluene and xylene; esters like ethyl acetate and isopropyl acetate; ethers such as ethyl ether, methyl t-butyl ether, di-isopropyl ether and tetrahydrofuran; amides such as formamide, dimethylforamide and N-methyl-pyrrolidone; nitriles such as acetonitrile and propionitrile; chlorinated hydrocarbons such as dichloromethane, ethylene dichloride and chloroform and mixtures thereof.

The estradiol divalerate (III) can also be obtained by the known methods in the literature by using coupling reagents such as ethyl chloroformate, DCC etc.

The process of step (i) is carried out at a temperature range of 30-120° C., preferably 50-100° C., most preferably 70-90° C.

The estradiol divalerate is crystallized from alcoholic solvents selected from the group of methanol, ethanol, propanol, isopropanol, butanol etc or mixtures thereof. The process for crystallization of estradiol divalerate (III) comprises of:
a) adding estradiol divalerate to alcoholic solvent,
b) heating the mixture to obtain a clear solution,
c) cooling the solution and
d) isolation of estradiol divalerate form A.

The process of step (iii) involves conversion of crystalline estradiol divalerate (III) to estradiol valerate (I) by treatment with reducing agent or alkali metal carbonates in water or mixture of organic solvents and water.

The alkali metal carbonates can be selected from potassium carbonate, sodium bicarbonate, sodium carbonate, cesium carbonate etc. Reducing agents can be selected from sodium borohydride, lithium aluminium hydride etc. The most preferred reagent for conversion of estradiol divalerate (iii) to estradiol valerate (I) is sodium borohydride.

The process of step (iii) of the present invention can be carried out in an organic solvent that include aromatic hydrocarbons like benzene, toluene and xylene; esters like ethyl acetate and isopropyl acetate; ethers such as ethyl ether, methyl t-butyl ether, di-isopropyl ether and tetrahydrofuran; amides such as formamide, dimethylforamide and N-methyl-pyrrolidone; nitriles such as acetonitrile and propionitrile; chlorinated hydrocarbons such as dichloromethane, ethylene dichloride and chloroform, alcohols such as methanol, ethanol, isopropanol, butanol and mixtures thereof. The most preferred solvent is methanol.

The process of step (iii) is carried out at a temperature range of 20-80° C. The preferred temperature range is 30-60° C.

The purity of estradiol valerate (I) obtained by the process of the present invention is ≧99.4% .

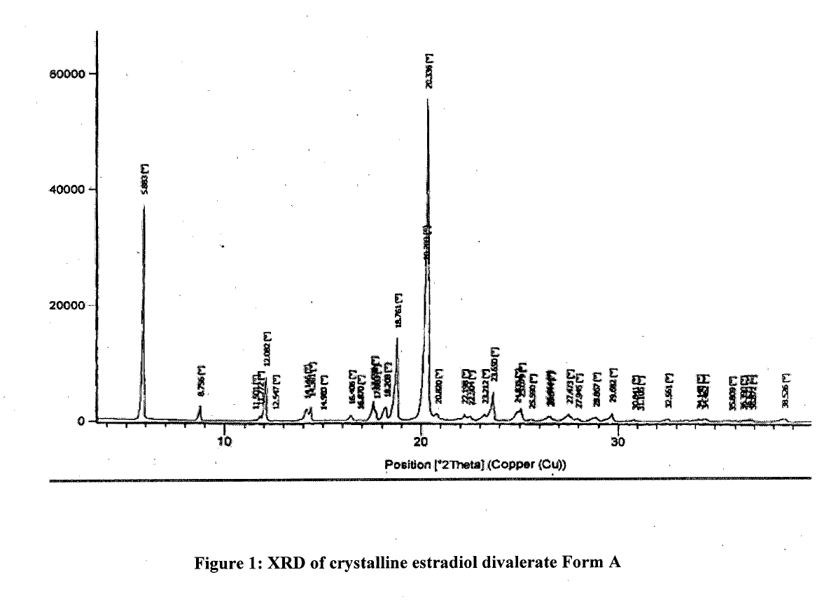

The present invention further provides novel crystalline form of estradiol divalerate referred as Form A. The crystalline Form A of estradiol divalerate obtained by the process of the present invention is characterized by XRPD pattern as shown in FIG. 1. The characteristic peaks in XRPD of estradiol divalerate Form A are as shown in table 1.

TABLE 1
XRPD of crystalline Form A of estradiol divalerate

| Degree 2 Theta | Relative Intensity |
|---|---|
| 5.88 | 66.80 |
| 8.75 | 4.66 |
| 12.08 | 13.47 |
| 14.14 | 3.56 |
| 14.38 | 4.20 |
| 16.40 | 1.75 |
| 17.55 | 6.17 |
| 17.68 | 3.57 |
| 18.20 | 3.93 |
| 18.76 | 25.65 |
| 20.20 | 46.76 |
| 20.33 | 100 |
| 23.64 | 8.85 |
| 25.07 | 4.20 |

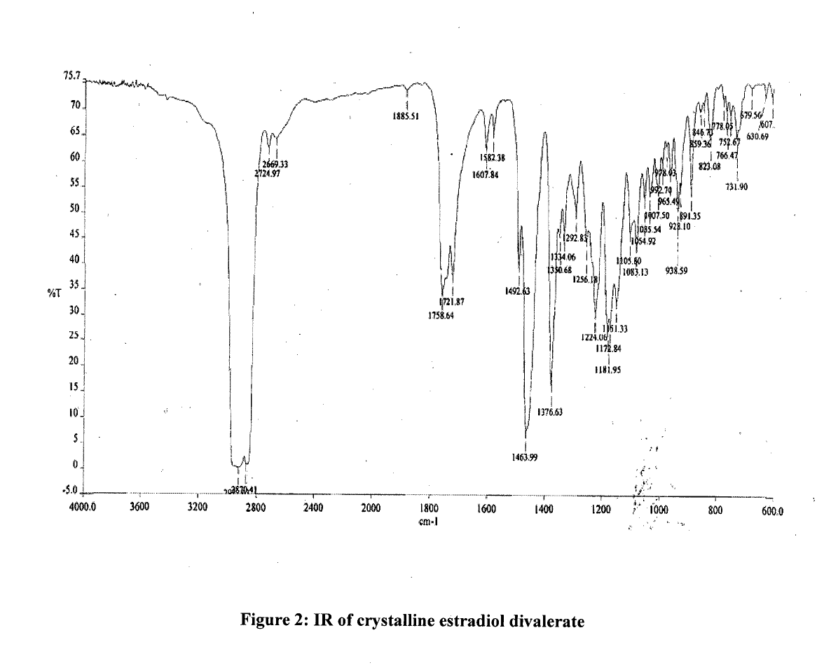

The crystalline estradiol divalerate Form A described herein is further identified by IR spectrum as shown in FIG. 2. The IR spectrum of crystalline estradiol divalerate Form A described herein has characteristic bands at 2870.41, 2724.97, 2669.33, 1885.51, 1721.87, 1758.64, 1582.38, 1607.84, 1492.63, 1463.99, 1376.63, 1350.68, 1334.06, 1292.83, 1256.18, 1224.06, 1172.84, 1151.33, 1181.95 cm⁻¹.

The melting point of the obtained crystalline estradiol divalerate Form A is in the range of 64-66° C.

The aforementioned process for the preparation of estradiol valerate (I) has the following advantages:
i) easy handling of the intermediate estradiol divalerate (III), which is obtained as crystalline solid,
ii) high purity of estradiol valerate (I),
iii) easy to scale up,
iv) economical process due to higher yields,
v) avoids oily intermediates and
vi) avoids laborious very high vaccum distillation techniques for purification of estradiol divalerate (III).

The principles, preferred embodiments, and modes of operation of the present invention have been described in the foregoing examples. The invention, which is intended to be protected herein, however, is not to be construed limited to the particular forms disclosed, since these are to be regarded as illustrative rather than restrictive. Variations and changes may be made by those skilled in the art, without departing from the scope of the invention.

### EXAMPLES

Step 1: Preparation of Estradiol Divalerate (III)

100 gm (0.367 moles) of Estradiol (II) was added in pyridine (500 ml) followed by addition of 217.2 ml (0.856 moles) of n-valeric anhydride. The reaction mixture was heated to 75-80° C. for 2 hours. The reaction mixture was cooled to room temperature and water (500 ml) was added followed by mixture of 1:1 hydrochloric acid-water (500 ml). Ethyl acetate (500 ml) was added and the layers were separated. The organic layer was washed with water (500 ml) followed by 6% sodium bicarbonate solution (500 ml). The organic layer was concentrated. Methanol (350 ml) was added to the residue and heated to get a clear solution. Cooled to 5-10° C. The solid was filtered, washed with methanol (100 ml) and dried under reduced pressure.

Yield: 94 gm (86.95%)

HPLC Purity: 96.86%

Step 2: Preparation of Estradiol Valerate (I)

100 gm (0.227 moles) of estradiol divalerate (III) was added to methanol (3500 ml) with stirring. Sodium borohydride (6.8 g) was added and heated to 40-45° C. for 2 hours. The reaction was cooled to 20-25° C. and to it water (900 ml) was added. The reaction mixture was further cooled to 10-15° C. and stirred for 1.5 hours. The solid was filtered, washed with 1:1 methanol-water mixture (200 ml) and dried under reduced pressure.

Yield: 90 gm (74%)

HPLC Purity: 99.64% and any other impurity ≦0.1%.

1. A process for preparation of estradiol valerate (I) which comprises of:
(i) reaction of estradiol (II) with n-valeric anhydride or n-valeryl chloride in the presence of base to obtain estradiol divalerate (III),
(ii) crystallization of estradiol divalerate (III) from alcoholic solvent to obtain crystalline estradiol divalerate (III) and
(iii) conversion of crystalline estradiol divalerate (III) to estradiol valerate (I).

2. The process of claim 1, wherein base used in step (a) is selected from group of organic or inorganic bases.

3. The process of claim 2 wherein inorganic base is selected from a group of carbonates or hydroxides of alkali earth metals.

4. The process of claim 2 wherein the organic base is selected from a group of pyridine, N-methyl morpholine, N-methyl pyrrolidine, tertiary alkyl amine such as triethyl amine, tertiary butyl amine.

5. The process of claim 4 wherein the most preferred base is pyridine.

6. The process of claim 1, wherein the step (a) is carried at a temperature range of 30-120° C.

7. The process of claim 6 wherein the preferred range of temperature is 70-90° C.

8. The process of crystallization of estradiol divalerate comprising the steps of:
a) adding estradiol divalerate to alcoholic solvent,
b) heating a mixture to obtain a clear solution,
c) cooling the solution and
d) isolation of estradiol divalerate form A.

9. The process of claim 8, wherein the solvent used in step (a) is selected from the group comprising of methanol, ethanol, propanol, isopropanol, butanol or mixtures thereof.

10. The process of claim 1, wherein in step (c), the conversion of crystalline estradiol divalerate (III) to estradiol valerate (I) is carried in the presence of reducing agent or alkali metal carbonates.

11. The process of claim 10, wherein the reducing agent is selected from the group of sodium borohydride, lithium aluminium hydride.

12. The process of claim 10, wherein alkali metal carbonate is selected from a group comprising of potassium carbonate, sodium bicarbonate, sodium carbonate, cesium carbonate.

13. The process of claim 1, wherein the solvent used in step (c) is selected from the group comprising of aromatic hydrocarbons like benzene, toluene and xylene, esters like ethyl acetate and isopropyl acetate, ethers such as ethyl ether, methyl tertiary butyl ether, diisopropyl ether and tetrahydrofuran, amides such as formamide, dimethylformamide and N-methyl pyrrolidine, nitriles such as acetonitrile and propionitrile, chlorinated hydrocarbons such as dichloromethane, ethylene dichloride and chloroform, alcohols such as methanol, ethanol, isopropanol, butanol and mixtures thereof.

14. The process according to claim 13, wherein the most preferred solvent is methanol.

15. Estradiol valerate (I) obtained by the process described in claim 1 having purity greater than 99.4%.

16. Novel crystalline form A of estradiol divalerate having 2 theta values at 5.88, 8.75, 12.08, 14.14, 14.38, 16.40, 17.55, 17.68, 18.20, 18.76, 20.20, 20.33, 23.64, 25.07.

17. Crystalline form A of estradiol divalerate according to claim 14, having XRPD as shown in FIG. 1.
